(19) United States
(12) Patent Application Publication
(10) Pub. No.: US 2019/0195881 A1
(43) Pub. Date: Jun. 27, 2019

(54) SENSITIVE DETECTION OF G-PROTEIN COUPLED RECEPTOR-ASSOCIATED SORTING PROTEIN 1 (GASP-1), GASP-1 MICROVESICLES, AND GASP-1 EXOSOMES

(71) Applicant: Proplex Technologies, LLC, Dresher, PA (US)

(72) Inventors: Frank N. Chang, Dresher, PA (US); George P. Tuszynski, Pittsgrove, NJ (US); Solomon Luo, Orwigsburg, PA (US); Jeff Yang, Bethesda, MD (US)

(73) Assignee: Proplex Technologies, LLC, Dresher, PA (US)

(21) Appl. No.: 16/227,642

(22) Filed: Dec. 20, 2018

Related U.S. Application Data
(60) Provisional application No. 62/608,962, filed on Dec. 21, 2017.

(51) Int. Cl. G01N 33/574

(52) U.S. Cl. CPC G01N 33/57492; G01N 2333/4719

(57) ABSTRACT

A highly sensitive method of detecting GASP-1 or a fragment thereof in a sample is provided. The method comprises (a) exposing a surface to a sample comprising GASP-1 or a fragment thereof; (b) immobilizing an anti-GASP-1 antibody to the surface in the presence of the GASP-1 or a fragment thereof; (c) measuring the amount of the anti-GASP-1 antibody immobilized to the surface; and (d) determining the presence of the GASP-1 or a fragment thereof in the sample based on the amount of the anti-GASP-1 antibody immobilized to the surface. A coating agent may be immobilized to the surface in step (a), and the anti-GASP-1 antibody may be immobilized to the surface via the coating agent. The coating agent may be selected from the group consisting of a conjugate of bovine serum albumin (BSA) and a GASP-1 peptide (BSA-GASP-1 conjugate) a capture antibody against a microvesicle or exosomal surface biomarker, and a combination thereof. Also provided are kits for detecting GASP-1 or its fragment.

### CROSS-REFERENCE TO RELATED APPLICATIONS

This application claims the benefit of U.S. Provisional Application No. 62/608,962, filed Dec. 21, 2017, the contents of which are incorporated herein by reference in their entireties for all purposes.

### FIELD OF THE INVENTION

The invention relates to a sensitive method for detecting G protein coupled receptor-associated sorting 1 (GASP-1), GASP-1 microvesicles, and GASP-1 exosomes at a very low concentration in a sample and applications thereof.

### BACKGROUND OF THE INVENTION

One way for detecting cancer is to use cancer biomarkers present on microvesicles or exosomes. Exosomes are smaller luminal vesicles (30-100 nm in diameter) originating from intracellular endosomes. Microvesicles are small membrane vesicles, (0.1-1 μm in diameter) released from cell membrane surface. In additional to blood, microvesicles and exosomes are present in other biological fluids, including saliva, urine, and cerebrospinal fluid.

Analysis of microvesicle or exosomal levels of circulating cancer biomarkers has been shown promising in cancer detection. Although several potential cancer biomarkers have been identified through proteomic analyses of cancer-derived microvesicles or exosomes from various types of cancers, none has been found suitable for early cancer detection.

One of the long-term goals for cancer research has been to discover a universal blood test that can detect the presence of cancer cells in an individual long before showing any cancer symptom. To be useful, new blood tests for cancer detection must have very high specificity; otherwise, too many healthy individuals will receive false positive test results, leading to unnecessary follow-up procedures and anxiety. Ideally, for a cancer test to be of practical use, it is desirable to have sensitivity of 90% or higher so that the false positive rate would be no more than 10%. Despite many efforts, no cancer biomarker has been reported to reach such high sensitivity and specificity for early cancer detection.

G protein coupled receptor-associated sorting 1 (GASP-1) is a useful biomarker for cancer detection as well as assessment of cancer progression and treatmentₛ but has not been shown useful for early cancer detection, especially in symptom-free individuals.

There remains a need for a highly sensitive and highly specific detection method to use GASP-1 as a biomarker for early cancer detection.

### SUMMARY OF THE INVENTION

The present invention provides a method of detection G coupled receptor-associated sorting 1 (GASP-1) or a fragment thereof in a sample and a related kit. The detection method is surprisingly sensitive and specific.

A method of detecting GASP-1 or a fragment thereof in a sample is provided. The method comprises (a) exposing a surface to a sample comprising GASP-1 or a fragment thereof; (b) immobilizing an anti-GASP-1 antibody to the surface in the presence of the GASP-1 or a fragment thereof; (c) measuring the amount of the anti-GASP-1 antibody immobilized to the surface; and (d) determining the presence of the GASP-1 or a fragment thereof in the sample based on the amount of the anti-GASP-1 antibody immobilized to the surface. A coating agent is immobilized to the surface, and the anti-GASP-1 antibody is immobilized to the surface via the coating agent. The coating agent is selected from the group consisting of a conjugate of bovine serum albumin (BSA) and a GASP-1 peptide (BSA-GASP-1 conjugate), a capture antibody against a microvesicle or exosomal surface biomarker, and a combination thereof.

The method may further comprise quantifying the concentration of the GASP-1 or a fragment thereof in the sample based on the amount of the anti-GASP-1 antibody immobilized to the surface and a standard curve of GASP-1 generated for the surface using the anti-GASP-1 antibody.

The method may further comprise immobilizing the coating agent to a surface such that the surface of step (a) is obtained.

In one embodiment, the coating agent is a BSA-GASP-1 conjugate, and step (c) comprises binding the anti-GASP-1 antibody specifically to the BSA-GASP-1 conjugate.

The GASP-1 peptide may consist of the amino acid sequence of

| | |
|---|---|
| EEASPEAVAGVGFESK, | (SEQ ID NO: 2) |

In another embodiment, the GASP-1 or a fragment thereof is on the surface of a microvesicle or an exosome in the sample, the coating agent is the capture antibody, step (b) comprises binding the microvesicle or exosome specifically to the capture antibody, and step (c) comprises binding the anti-GASP-1 antibody specifically to the GASP-1 or a fragment thereof.

The microvesicle or exosomal surface biomarker may be selected from the group consisting of CD9, CD63, CD81 and a combination thereof.

The sample may comprise 0.01-0.02 ng/ml of the GASP-1 or a fragment thereof.

The anti-GASP-1 antibody may be selected from the group consisting of a recombinant monoclonal antibody, a recombinant polyclonal antibody, a humanized antibody and an antigen-binding fragment thereof.

The GASP-1 or a fragment thereof in the sample may be from a biological fluid of a subject, and the method may further comprise determining the concentration of the GASP-1 or a fragment thereof in the biological fluid in the subject. The biological fluid may comprise 0.01-0.02 ng/ml of the GASP-1 or a fragment thereof. The biological fluid may be selected from the group consisting of plasma, serum, saliva, urine, and cerebrospinal fluid. For example, the biological fluid may be serum. The subject may be free of a cancer symptom or may have cancer.

The subject may have received a treatment of cancer, and the biological fluid may be in the subject after the treatment. The method may further comprise determining a concentration of the GASP-1 or a fragment thereof in a biological fluid in the subject before the treatment. The method may further comprise comparing the concentration of the GASP-1 or a fragment thereof in the biological fluid in the subject after the treatment with the concentration of the GASP-1 or a fragment thereof in the biological fluid in the subject before the treatment.

The cancer may be selected from the group consisting of breast cancer, bladder cancer, ovarian cancer, colon cancer, endometrial carcinoma, esophagus squamous cell carcinoma, glioma, head cancer, neck cancer, hepatocellular carcinoma, infiltrating ductal breast carcinoma, larynx cancer, lung cancer, melanoma, mucinous cystadenocarcinoma of ovary, pancreatic cancer, prostate cancer, renal cell carcinoma, small bowel malignant stromal tumor, and stomach adenocarcinoma. The cancer may be selected from the group consisting of breast cancer, prostate cancer, lung cancer, pancreatic cancer, liver cancer and glioblastoma.

The method may further comprise comparing the concentration of the GASP-1 or a fragment thereof in the biological fluid in the subject with a concentration of the GASP-1 or a fragment thereof in a control biological fluid in a symptom-free individual.

Where the subject is suspected of having prostate cancer, the method may further comprise comparing the concentration of the GASP-1 or a fragment thereof in the biological fluid in the subject with a concentration of the GASP-1 or a fragment thereof in a biological fluid in an individual having benign prostatic hyperplasia (BPH). The subject may be tested positive in a prostate specific antigen (PSA) test.

Where the subject is suspected of having stage IV invasive breast cancer, the method may further comprise comparing the concentration of the GASP-1 or a fragment thereof in the biological fluid in the subject with a concentration of the GASP-1 or a fragment thereof in a biological fluid in an individual having inflammatory breast cancer.

The method may further comprise detecting a cancer biomarker in the sample. The cancer biomarker may be selected from the group consisting of CA125, CA19-9, CA15-3, CEA and a combination thereof.

A kit comprising a surface and an anti-GASP-1 antibody is provided. A coating agent is immobilized to the surface. The coating agent is selected from the group consisting of a conjugate of bovine serum albumin (BSA) and a GASP-1 peptide (BSA-GASP-1 conjugate), a capture antibody against a microvesicle or exosomal surface biomarker, and a combination thereof. The kit may further comprise an instruction for carrying out the method of detecting GASP-1 or a fragment in a sample according to the present invention.

A kit comprising a surface, a coating agent and air anti-GASP-1 antibody is also provided. The coating agent is selected from the group consisting of a conjugate of bovine serum albumin (BSA) and a GASP-1 peptide (BSA-GASP-1 conjugate), a capture antibody against a microvesicle or exosomal surface biomarker, and a combination thereof. The kit may further comprise a first instruction for immobilizing the coating agent to the surface and a second instruction for carrying out the method of detecting GASP-1 or a fragment in a sample according to the present invention.

The coating agent may be the BSA-GASP-1 conjugate.

In the kit, the GASP-1 peptide may consist of the amino acid sequence of

| | |
|---|---|
| EEASPEAVAGVGFESK, | (SEQ ID NO: 2) |

In the kit, the coating agent may be the capture antibody.

In the kit, the microvesicle or exosomal surface biomarker may be selected from the group consisting of CD9, CD63, CD81 and a combination thereof.

In the kit, the anti-GASP-1 antibody may be biotinylated.

### BRIEF DESCRIPTION OF THE DRAWINGS

FIG. 1 shows the distribution of GASP-1 in pancreatic cells in a pancreatic tissue from a symptom-free individual as shown by immunohistochemical (IHC) staining with an anti-GASP-1 polyclonal antibody.

FIG. 2 shows the distribution of GASP-1 in normal pancreatic cells (left panel) or pancreatic ductal adenocarcinoma (PDAC) cells (right panel). GASP-1 was visualized in microvesicles and exosomes in the PDAC cells, but not in the normal pancreatic cells.

FIG. 3 shows GASP-1 microvesicles and exosomes in an enlarged image of a section from the right panel of FIG. 2. Numerous microvesicles and exosomes were observed, and the arrows indicate some of the microvesicles and exosomes.

FIG. 4 shows GASP-1 microvesicles In the process of being released from pancreatic adenocarcinoma cells. Some released microvesicles and exosomes were observed.

FIG. 5 shows a representative standard curve generated for determining GASP-1 concentrations using a competitive ELISA.

FIG. 6 shows GASP-1 concentrations in serum samples from symptom-free (normal) individuals or breast cancer patients.

FIG. 7 shows GASP-1 concentrations in serums les from symptom-free (normal) individuals or breast cancer patients.

FIG. 8 shows sensitivity and specificity of the competitive ELISA used to generate the results in FIG. 7. The Area under ROC curve (AUC) number was 1.00.

FIG. 9 shows GASP-1 concentrations in serum samples from symptom-free (normal) individuals, patients with inflammatory breast cancer or patients with Stage IV invasive breast cancer.

FIG. 10 shows GASP-1 concentrations in serum samples from symptom-free (normal) individuals or prostate cancer patients.

FIG. 11 shows GASP-1 concentrations in serum samples from symptom-free (healthy normal) individuals, age-matched symptom-free (age matched normal) individuals, benign prostatic hyperplasia patients (benign BPH) or prostate cancer patients (prostate Ca).

FIG. 12 shows GASP-1 concentrations in serum samples from symptom-free (normal) individuals or lung cancer patients.

FIG. 13 shows sensitivity and specificity of a competitive ELISA to determine GASP-1 concentrations in serum samples from symptom-free (normal) individuals or pancreatic cancer patients. The AUC number was 0.9824.

FIG. 14 shows sensitivity and specificity of a competitive ELISA used to determine GASP-1 concentrations in serum samples from symptom-free (normal) individuals or liver cancer patients. The AUC number was 0.9812.

FIG. 15 shows sensitivity and specificity of a competitive ELISA used to determine GASP-1 concentrations in serum samples from symptom-free (normal) individuals or glioblastoma cancer patients. The AUG number was 1.00.

### DETAILED DESCRIPTION OF THE INVENTION

The present invention provides a highly sensitive and highly specific method of detecting G protein coupled receptor-associated sorting 1 (GASP-1) in a sample at a very low concentration and uses of this method for cancer detection, especially early cancer detection, and assessment of cancer progression and cancer treatment.

GASP-1 is a cytosolic protein, which is highly overexpressed in many cancers, including breast, brain, liver and lung cancers. In the cytosol, GASP-1 interacts with the terminal end of many G protein coupled receptors (GPCRs). Through its binding to different GPCRs, GASP-1 functions as a signal transduction silencer by directing the ligand-bound GPCRs via “sorting endosomes” to lysosomes for destruction. However, when GASP-1 is overexpressed, this sorting process is inhibited and the ligand-bound GPCRs together with GASP-1 stay in the endosomes (“recycling endosomes”). After multivesicular bodies, a subset of the recycling endosomes, fuse with plasma membrane, intraluminal vesicles are formed and their contents are released as exosomes. Some recycling endosomes may fuse directly with plasma membrane, and become part of the plasma membrane before being released as microvesicles.

The invention is based on the inventors' surprising discovery of GASP-1 expression in symptom-free individuals and circulating GASP-1 in cancer patients, as well as GASP-1 containing microvesicles and GASP-1 containing endosomes released from cancer cells. For detection of various cancers, the method of the present invention has been found to have unexpectedly high sensitivity of, for example, as low as 0.01 ng/ml of the GASP-1 or its fragment, and unexpectedly high specificity of, for example, at least 98%.

The terms “symptom-free,” “normal” and “control” are used interchangeably and refer to an individual who has not shown any cancer symptom. The symptom-free individual may have been tested positive or negative with a cancer biomarker. The symptom-free individual may have cancer cells. The symptom-free individual may be predisposed to cancer.

The term “cancer patient” used herein refers an individual who has shown at least one cancer symptom. The cancer patient may have been tested positive or negative with a cancer biomarker. The cancer patient has cancer cells. The cancer patient may have received a cancer treatment.

The present invention provides a method of detecting G protein coupled receptor-associated sorting 1 (GASP-1) or its fragment in a sample. The method comprises exposing a surface to a sample comprising GASP-1 or its fragment; immobilizing an anti-GASP-1 antibody to the surface in the presence of the GASP-1 or its fragment; measuring the amount of the anti-GASP-1 antibody immobilized to the surface; and determining the presence of the GASP-1 or a fragment thereof in the sample based on the amount of the anti-GASP-1 antibody immobilized to the surface.

A coating agent may be immobilized to the surface. The coating agent may be selected from the group consisting of a conjugate of a protein, for example, bovine serum albumin (BSA) or casein, and a GASP1 peptide (protein-GASP-1 conjugate), a capture antibody against a microvesicle or exosomal surface biomarker, and a combination thereof. The anti-GASP-1 antibody may be immobilized to the surface via the coating agent.

The method may further comprise immobilizing the coating agent to a surface such that the surface used for exposing to the sample is obtained. Before the exposure, the coated surface may be blocked by a blocking agent, for example, a BSA solution, to block sites on the surface not covered by the coating agent. The coated and blocked surface may be washed before the exposure.

The method may further comprise quantifying the concentration of the GASP-1 or fragment thereof in the sample based on the amount of the anti-GASP-1 antibody immobilized to the surface and a standard curve of GASP-1 generated for the surface using the anti-GASP-1 antibody. The GASP-1 standard curve may be generated using a series of solutions comprising GASP-1 at different concentrations. The method may further comprise generating the GASP-1 standard curve.

The coating agent may be a protein-GASP-1 conjugate. The protein may be BSA and the corresponding conjugate may be a BSA-GASP-1 conjugate. The protein may be casein, and the corresponding conjugate may be a casein-GASP-1 conjugate. The method may further comprise binding the anti-GASP-1 antibody specifically to the protein-GASP-1 conjugate, for example, the BSA-GASP-1 conjugate. The GASP-1 peptide may be any fragment of GASP-1 (SEQ ID NO: 1), preferably any GASP-1 peptide known to be exposed on the surface of a cell, a microvesicle or an exosome. For example, the GASP-1 peptide may comprise an amino acid sequence selected from the group consisting of EEASPEAVAGVGFESK (SEQ ID NO: 2), CSKSSPKAEEEEV (SEQ ID NO: 3), FWDGKEVSEEAGPC (SEQ ID NO: 4), and a combination thereof.

In one embodiment, the GASP-1 peptide consists of the amino acid sequence of

| | |
|---|---|
| EEASPEAVAGVGFESK, | (SEQ ID NO: 2) |

The coating agent may be a capture antibody. The GASP-1 or a fragment thereof may be on the surface of a microvesicle or an exosome in the sample. The method may further comprise binding the microvesicle or exosome specifically to the capture antibody, and/or binding the anti-GASP-1 antibody specifically to the GASP-1 or its fragment. The microvesicle or exosomal surface biomarker may be selected from the group consisting of CD9, CD63, CD81, and a combination thereof. The microvesicle or exosomal surface biomarker may be selected from other reported exosomal proteins such as Glypican-1 (GPC1), CD24, EpCAM, CA125, PSA, CEA, and Her2.

The anti-GASP-1 antibody may be biotinylated. The anti-GASP-1 antibody may bind specifically to the peptide of EEASPEAVAGVGFESK (SEQ ID NO: 2). The anti-GASP-1 antibody may be any antibody capable of binding to GASP-1 or its fragment.

The anti-GASP-1 antibody may be a recombinant antibody or an antigen-binding fragment thereof. The antibody may be a monoclonal antibody, a polyclonal antibody or a humanized antibody.

GASP-1 or its fragment may be in a cell, for example, in the cytoplasm or cell membrane of the cell, or outside of a cell. GASP-1 or its fragment may be associated with a microvesicle or an exosome, for example, or on the surface of a microvesicle or an exosome, or not associated with a microvesicle or an exosome. An exosome associated with GASP-1 or its fragment is a GASP-1 exosome. A GASP-1 exosome may be in a cell before it is released, for example, in the cytoplasm or in the cell membrane of the cell, or outside of a cell. A microvesicle associated with GASP-1 or its fragment is a GASP-1 microvesicle. A GASP-1 microvesicle may be on the cell membrane or outside the cell.

The sample may comprise about 0.001-1.000, 0.005-0.100, 0.005-0.05, 0.005-0.02, 0.01-0.05, 0.01-0.02 or 0.010-0.015 ng/ml of the GASP-1 or a fragment thereof. The sample may comprise at least about 0.001, 0.005, 0.01, 0.02, 0.05, 0.1, 0.2, 0.5, 1, 2, 5, 10, 20, 50, 100, 200, 500 or 1000 ng/ml of the GASP-1 or its fragment. The sample may comprise no more than about 10, 5, 1, 0.5, 0.1, 0.05, 0.02, 0.01, 0.005, 0.002 or 0.001 ng/ml of the GASP-1 or its fragment.

The GASP-1 or its fragment in the sample may be from a biological fluid of a subject. The method may further comprise determining the concentration of the GASP-1 or a fragment thereof in the biological fluid in the subject. The biological fluid may be selected from the group consisting, of plasma, serum, saliva, urine, and cerebrospinal fluid. The biological fluid may be serum.

The biological fluid may comprise about 0.001-1.000, 0.005-0.100, 0.005-0.05, 0.005-0.02, 0.01-0.05, 0.01-0.02 or 0.010-0.015 ng/ml of the GASP-1 or a fragment thereof. The biological fluid may comprise at least about 0.001, 0.005, 0.01, 0.02, 0.05, 0.1, 0.2, 0.5, 1, 2, 5, 10, 20, 50, 100, 200, 500 or 1000 ng/ml of the GASP-1 or its fragment. The biological fluid may comprise no more than about 10, 5, 1, 0.5 0.1, 0.05, 0.02, 0.01, 0.005, 0.002 or 0.001 ng/ml of the GASP-1 or its fragment.

The GASP-1 or its fragment may have been in the circulatory system of a subject. The term “circulatory system” used herein refers to the system that circulates blood and lymph through the body of the subject, consisting of the heart, blood vessels, blood, lymph, and the lymphatic vessels and glands. In the circulatory system, GASP-1 or its fragment may be associated with a microvesicle or an exosome, for example, in or on a microvesicle or an exosome. A microvesicle associated with a GASP-1 or its fragment is a GASP-1 microvesicle,.A GASP-1 microvesicle may be on cell membrane. A GASP-1 microvesicle may be released and outside of a cell. An exosome associated with a GASP-1 or its fragment is a GASP-1 exosome. A GASP-1 exosome may be in a cell before it is released, for example, in the cytoplasm or in the cell membrane of the cell. A GASP-1 exosome may be outside of a cell. Alternatively, GASP-1 or its fragment in the circulatory system may not be associated with a microvesicle, an exosome or a cell, and is deemed to free.

The subject may be a mammal, preferably a human. The subject may be free of a cancer symptom. The subject may nave cancer or may be predisposed to cancer. The cancer may be selected from the group consisting of breast cancer, bladder cancer, ovarian cancer, colon cancer, endometrial carcinoma, esophagus squamous cell carcinoma, glioma, head cancer, neck cancer, hepatocellular carcinoma, infiltrating, ductal breast carcinoma, larynx cancer, lung cancer, melanoma, mucinous cystadenocarcinoma of ovary, pancreatic cancer, prostate cancer, renal cell carcinoma, small bowel malignant stromal tumor, and stomach adenocarcinoma. Preferably, the cancer is associated with elevated GASP-1 expression. For example, the cancer may be selected from the group consisting of breast cancer, prostate cancer, lung cancer, pancreatic cancer, liver cancer and glioblastoma.

The presence of GASP-1 or its fragment in a test biological fluid of a test subject may indicate that the test subject has cancer. The cancer may be associated with elevated GASP-1 expression (or overexpression of GASP-1). For example, a test biological fluid of a test subject having at least about 0.001, 0.005, 0.01, 0.02, 0.05, 0.1, 0.2, 0.5, 1, 2, 5, 10, 20, 50, 100, 200, 500 or 1000 ng/ml of the GASP-1 or its fragment may indicate that the test subject has cancer. A test biological fluid of a test subject having a concentration of GASP-1 or its fragment at least about 5, 10, 50, 100, 500 or 1000 times higher than that in a control biological fluid in a symptom-free individual may indicate that the test subject has cancer. The control biological fluid and the test biological fluid are preferably from the same tissue type. The test subject may be the symptom-free individual, and the control biological fluid and the test biological fluid may be from the same tissue.

The presence of microvesicles or exosomes associated with GASP-1 or its fragment in a test biological fluid of a test subject may suggest that the test subject have cancer. The cancer may be associated with elevated GASP-1 expression (or overexpression of GASP-1). For example, a test biological fluid of a test subject having at least about 0.001, 0.005, 0.01, 0.02, 0.05, 0.1, 0.2, 0.5, 1, 2, 5, 10, 20, 50, 100, 200, 500 or 1000 ng/ml of the GASP-1 or its fragment associated with the microvesicles or exosomes may indicate that the test subject has cancer. A test biological fluid of a test subject having a concentration of GASP-1 or its fragment associated with the microvesicles or exosomes at least about 5, 10, 50, 100, 500 or 1000 times higher than a control biological fluid of a symptom-free individual may indicate that the test subject has cancer. The control biological fluid and the test biological fluid are preferably from the same tissue type. The test subject may be the symptom-free individual, and the control biological fluid and the test biological fluid may be from the same tissue.

The method may further comprise comparing the concentration of the GASP-1 or a fragment thereof in the biological fluid in the subject with a concentration of the GASP-1 or a fragment thereof in a biological fluid in a symptom-free individual. The biological fluid of the symptom-free individual may have GASP-1 expression at a minimal level, for example, no more than about 10, 5, 1, 0.5, 0.1, 0.05, 0.02, 0.01, 0.005, 0.002 or 0.001 ng/ml of the GASP-1 or its fragment.

The subject may have received a cancer treatment. The biological fluid may be in the subject after the treatment. The cancer may be associated with elevated GASP-1 expression (or overexpression of GASP-1). The method may further comprise determining a concentration of the GASP-1 or its fragment in a biological fluid in the subject before the treatment. The method may further comprise comparing the concentration of the GASP-1 or its fragment in the biological fluid in the subject after the treatment with the concentration of the GASP-1 or a fragment thereof in the biological fluid in the subject before the treatment. A change in the concentration of the GASP-1 or its fragment in the biological fluid in the subject after the treatment may be used to assess the efficacy of the treatment. A reduction of the concentration of the GASP-1 or its fragment in the biological fluid in the subject after the treatment by, for example, at least about 5, 10, 50, 100, 500 or 1000 times may indicate that the treatment is effective.

If the subject is suspected of having prostate cancer, the method may further comprise comparing the concentration of the GASP-1 or a fragment thereof in the biological fluid in the subject with a concentration of the GASP-1 or a fragment thereof in a biological fluid in an individual having benign prostatic hyperplasia (BPH). A higher concentration of the GASP-1 or a fragment thereof in a biological fluid in a subject suspected of having prostate cancer than that in a biological fluid in an individual having benign prostatic hyperplasia (BPH) by, for example, at least about 5, 10, 50, 100, 500 or 1000 times, may indicate that the subject has prostate cancer. The subject may be tested positive or negative in a prostate specific antigen (PSA) test. In one embodiment, the subject is tested false positive in a PSA test. This method is useful for differentiating prostate cancer from BPH in a subject.

If the subject is suspected of having stage IV invasive breast cancer, the method may further comprise comparing the concentration of the GASP-1 or a fragment thereof in the biological fluid in the subject with a concentration of the GASP-1 or a fragment thereof in a biological fluid in an individual having inflammatory breast cancer. A concentration of the GASP-1 or a fragment thereof in a biological fluid in a subject suspected of having stage IV invasive breast cancer higher than that in a biological fluid in an individual having inflammatory breast cancer by, for example, at least about 5, 10, 50, 100, 500 or 1000 times, may indicate that the subject has stage IV invasive breast cancer. This method is useful for differentiating stable IV invasive breast cancer from inflammatory breast cancer in a subject.

The detection method of the present invention may further comprise detecting a cancer biomarker in the sample. The cancer biomarker is not GASP-1, and may be specific for breast cancer, bladder cancer, ovarian cancer, colon cancer, endometrial carcinoma, esophagus squamous cell carcinoma, glioma, head cancer, neck cancer, hepatocellular carcinoma, infiltrating ductal breast carcinoma, larynx cancer, lung cancer, melanoma, mucinous cystadenocarcinoma of ovary, pancreatic cancer, prostate cancer, renal cell carcinoma, small bowel malignant stromal tumor, and stomach adenocarcinoma. For example, the cancer may be selected from the group consisting of breast cancer, prostate cancer, lung cancer, pancreatic cancer, liver cancer or glioblastoma. Examples of the cancer biomarkers include CA125, CA19-9, CA15-3, and CEA.

The detection method of the present invention is useful for diagnosis and/or assessment of cancer associated with elevated GASP-1 expression (or overexpression of GASP-1). This method may be used for cancer detection in a subject, especially early cancer in a symptom-free subject, based on the expression level of GASP-1 or its fragment in a biological fluid sample from the subject. This method may be used to assess cancer treatment or cancer progression in a subject based on the expression level of GASP-1 or its, fragment in a biological fluid sample from the subject. Exemplary cancer includes breast cancer, bladder cancer, ovarian cancer, colon cancer, endometrial carcinoma, esophagus squamous cell carcinoma, glioma, head cancer, neck cancer, hepatocellular carcinoma, infiltrating ductal breast carcinoma, larynx cancer, lung cancer, melanoma, mucinous cystadenocarcinoma of ovary, pancreatic cancer, prostate cancer, renal cell carcinoma, small bowel malignant stromal tumor, and stomach adenocarcinoma. The detection method may provide high sensitivity of, for example, at least about 90%, 95%, 98%, 99% or 99.9%. In one embodiment, the sensitivity of the method may be 100%. The detection method may provide high specificity of, for example, at least about 90%, 95%, 98%, 99% or 99.9%. In one embodiment, the specificity of the method may be 100%.

The present invention also provides a kit comprising a surface and an anti-GASP-1 antibody. A coating agent is immobilized to the surface and is selected from the group consisting of a conjugate of a protein, for example, bovine serum albumin (BSA) or casein, and a GASP-1 peptide (protein-GASP-1 conjugate), a capture antibody against a microvesicle or exosomal surface biomarker, and a combination thereof. The kit may further comprise an instruction for carrying out the method of the present invention.

The present invention further provides a kit comprising a surface, a coating agent and an anti-GASP-1 antibody. The coating agent is selected from the group consisting of a conjugate of a protein, for example, bovine serum albumin (BSA) or casein, and a GASP-1 peptide (protein-GASP-1 conjugate), a capture antibody against a microvesicle or exosomal surface biomarker, and a combination thereof. The kit may further comprise a first instruction for immobilizing the coating agent to the surface and a second instruction for carrying out the method of the present invention.

The coating agent in the kit may be a protein-GASP-1 conjugate, for example, a BSA-GASP-1 conjugate. The GASP-1 peptide may comprise the amino acid sequence of EEASPEAVAGVGFESK (SEQ ID NO: 2). In one embodiment, the GASP-1 peptide consists of the amino acid sequence of EEASPEAVAGVGFESK (SEQ ID NO: 2).

The coating agent in the kit may be a capture antibody. The microvesicle or exosomal surface biomarker may be selected from the group consisting of CD9, CD63, CD81 and a combination thereof.

The anti-GASP-1 antibody in the kit may be biotinylated. The anti-GASP-antibody may bind specifically to the peptide of EEASPEAVAGVGFESK (SEQ ID NO: 2). The anti-GASP-1 antibody may be any antibody capable of binding to GASP-1 or its fragment. The anti-GASP-1 antibody may be a recombinant antibody or an antigen-binding fragment thereof. The antibody may be a monoclonal antibody, a polyclonal antibody or a humanized antibody. The anti-GASP-1 antibody may bind specifically to the peptide of EEASPEAVAGVGFESK (SEQ ID NO: 2).

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### EXAMPLE 1. GASP-1 OVEREXPRESSION IN CANCER CELLS AND THEIR RELEASE IN MICROVESICLES OR EXOSOMES

The distribution of GASP-1 in pancreatic cells was visualized by immunohistochemical (IHC) staining with an anti-GASP-1 polyclonal antibody raised against a GASP-1 fragment consisting of EEASPEAVAGVGFESK (SEQ ID NO: 2). The intensity of the staining indicates the GASP-1 expression level.

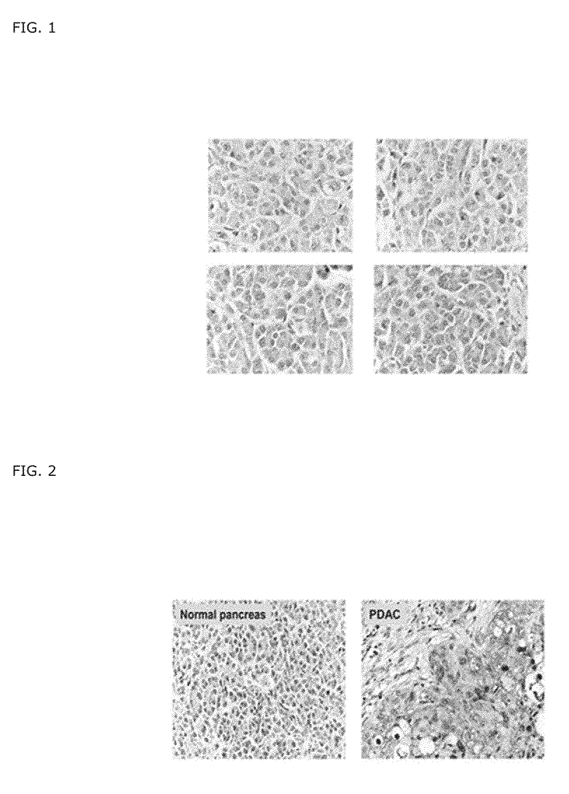

GASP-1 overexpression was surprisingly found in some pancreatic cells from a symptom-free individual. Sections of a pancreatic tissue from the symptom-free individual were stained with the anti-GASP-1 antibody. GASP-1 expression was found at a minimal level in some pancreatic cells (FIG. 1, upper left and right panels), but at a high GASP-1 expression level in other cells (FIG. 1, lower left panel). Some GASP-1 expressing cells showed intense GASP-1 cytosol staining and some staining on cell surface (FIG. 1, lower right panel), suggesting that in some pancreatic cells the overexpressed GASP-1 has migrated to cell surface but not released from the cells. Thus, as shown in FIG. 1, GASP-1 was expressed progressively in some pancreatic cells from the symptom-free individual.

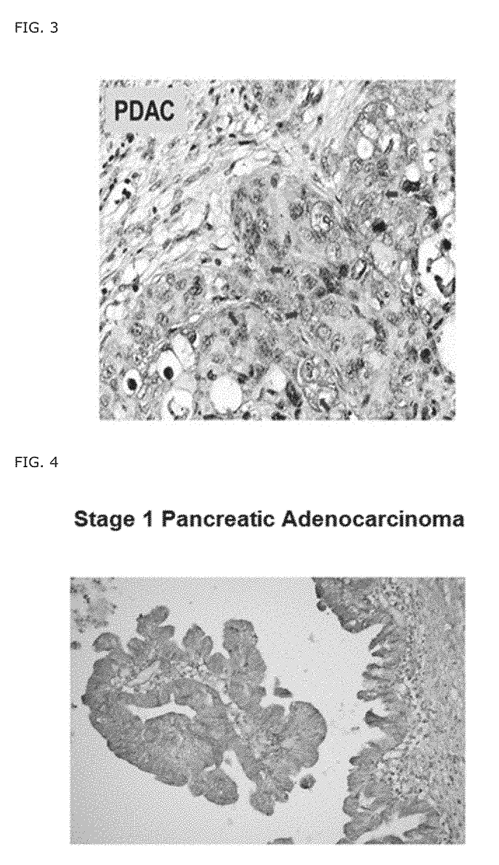

The GASP-1 overexpression was observed in pancreatic ductal adenocarcinoma (PDAC), but not normal pancreatic cells. The cell samples were is kindly supplied by Dr. Leland Chung of Cedars-Sinai Medical Center, Los Angeles Calif. The normal pancreatic cells showed GASP-1 expression at a minimal level (FIG. 2, left panel) while microvesicles with GASP-1 expression (GASP-1 microvesicles) and exosomes with GASP-1 expression (GASP-1 exosomes) were observed outside and near the PDAC cells (FIG. 2 right panel). A close-up view of the PDAC cell membrane in FIG. 2 shows a rather rough PDAC cell membrane surface (FIG. 3), suggesting that a huge amount of GASP-1 in cancer cell membrane was waiting to be released from the cancer cells as GASP-1 microvesicles. The GASP-1 microvesicles and GASP-1 exosomes outside and near the PDAC cells appeared to have been released from the PDAC cells. FIG. 4 shows that GASP-1 microvesicles were in the process of being released near the tip of pancreatic cancer cell membrane by budding from the membrane. FIG. 4 also shows released GASP-1 microvesicles and GASP-1 exosomes. Thus, as shown in FIGS. 2-4, pancreatic cancer cells overexpressed GASP-1 in microvesicles and GASP-1 exosomes, and both the GASP-1 microvesicles and GASP-1 exosomes are released from the cancer ceils.

The unexpected findings of the progressively high production of GASP-1 inside a symptom-free individual's pancreatic tissue cells and the accumulation of GASP-1 microvesicles on the surface of pancreatic cancer cells before they are released suggest a correlation between cancer initiation and GASP-1 overexpression, and GASP-1 may be an excellent early detection biomarker for pancreatic cancer.

Because the over-expressed GASP-1 by cancer cells appear to have been released from the cancer cells as microvesicles (GASP-1 microvesicles) or exosomes (GASP-1 exosomes), the GASP-1 in circulation (e.g., blood) may be either freed from the microvesicles or exosomes or remain on the surface of the microvesicles exosomes. The continuing increase in the number of GASP-1 microvesicles or GASP-1 exosomes during cancer progression suggests that GASP-1 microvesicles, or GASP-1 exosomes could also be used as a cancer biomarker to monitor cancer progression. Thus, an increase in GASP-1 microvesicles or GASP-1 exosome quantity in cancer cells and altered cargo expression may be considered as a potent biomarker for alteration of normal physiological states.

### EXAMPLE 2. HIGHLY SENSITIVE GASP-1 COMPETITIVE ELISA

A highly sensitive GASP-1 competitive ELISA procedure was developed to detect and quantify GASP-1 or a fragment thereof in a sample. In particular, a conjugate of BSA (bovine serum albumin) and a GASP-1 peptide (BSA-GASP-1 conjugate) was used to coat a single plate to retain the GASP-1 peptide before detecting GASP-1 or a fragment thereof in a sample. The sequence of the GASP-1 peptide was EEASPEAVAGVGFESK (SEQ ID NO: 2).

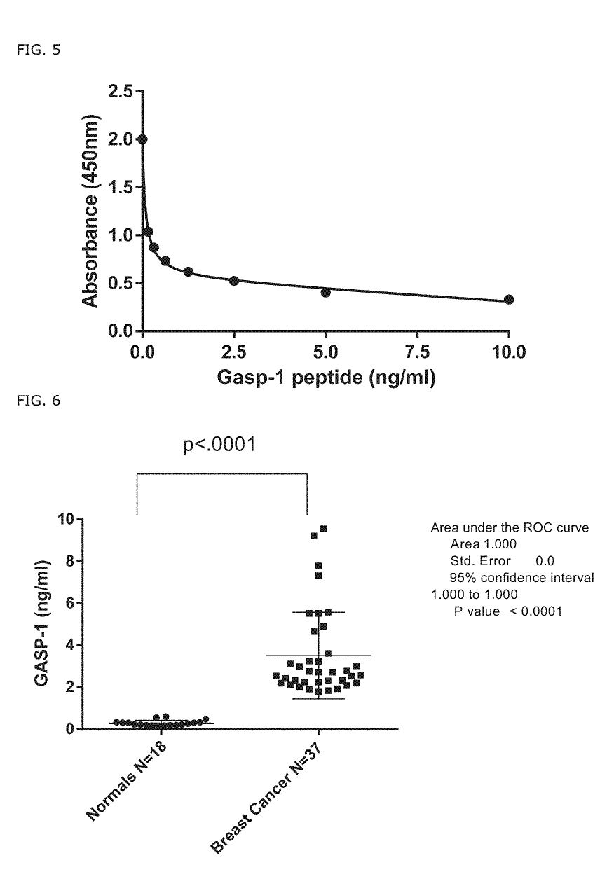

An ELISA plate having multiple wells was coated with 100 μl of a solution containing the BSA-GASP-1 conjugate either overnight (e.g., 8-12 hours) with shaking or for 2 hours at room temperature. This was followed by the addition of 300 μl of 1% BSA solution and incubation for 20 min to block sites on the plate not covered by the BSA-GASP-1 conjugate. The solution was aspirated and washed once with Tris (tris-hydroxymethyl aminomethane)-buffed saline solution containing 0.05% Tween 20 (TBST). 50 μl of a diluted serum sample (or a standard GASP-1 peptide solution for standard curve construction) was added to wells of the plate. This was followed by the additional of 50 μl of 1:8000 biotinylated anti-GASP-1 antibody against the peptide of SEQ ID NO: 2 to the wells and incubation for 2 hours at room temperature with shaking. The final dilution in the well was 1:16,000 for the antibody and 1:8 for the sample in a volume of 100 μl. The plate was then aspirated and washed 3 times with 300 μl of TBS-Tween buffer (TBST). The TBST solution was then aspirated before 100 μl of diluted Streptavidin-HRP was added and incubated for 30 min to one hour at room temperature. The mixture was then aspirated and the plate was washed 3 times with 300 μl of TBST buffer. The TBST buffer was then aspirated before 100 μl of TMB (tetramethylbenzidine) solution was added and incubated with shaking for 10 to 30 min (stop the solution if the control well was highly blue) for color development. 50 μl of 1 N sulfuric acid was added to each well to stop the color development. The absorbance of wells was read at 450 nm and used to calculate the concentration of the GASP-1 or a fragment thereof. Ideally, the absorbance reading for a control well (without a competing peptide) should be around 1.2. Slightly higher or lower readings are acceptable. A representative standard curve is shown in FIG. 5. The sensitivity of this competitive ELISA could be 0.01 ng/ml.

### EXAMPLE 3. GASP-1 SANDWICH ELISA

A sandwich ELISA can be developed to detect and quantify GASP-1 microvesicles or GASP-1 exosomes in a sample using two different antibodies, a capture antibody that binds specifically to the microvesicles or exosomes and a detection antibody against GASP-1. The sandwich ELISA procedure will be similar to the competitive ELISA procedure as described in Example 2 except that the capture antibody will be immobilized to a surface to capture GASP-1 microvesicles or GASP-1 exosomes in a sample before using an anti-GASP-1 antibody to detect GASP-1 on a microvesicle or an exosome bound to the surface via the capture antibody. The microvesicle or exosomal surface markers may be selected from CD9, CD63, CD 81 and a combination thereof. These proteins are known to be present on the surface of both microvesicles and exosomes. Once captured, GASP-1 microvesicles or GASP-1 exosomes will be specifically detected using biotinylated anti-GASP-1 antibody, allowed by Streptavidin-HRP and color detection.

A sandwich ELISA can also be developed to detect and quantify GASP-1 in a sample using two different antibodies, a capture antibody and a detection antibody that bind specifically to two different exposed peptides in GASP-1. Each GASP-1 peptide may comprise an amino acid sequence selected from the group consisting of EEASPEAVAGVGFESK (SEQ ID NO: 2), CSKSSPKAEEEEV (SEQ ID NO: 3), FWDGKEVSEEAGPC (SEQ ID NO: 4), and a combination thereof. One of the two antibodies may consist of SEQ ID NO: 2.

### EXAMPLE 4. HIGH SENSITIVITY AND SPECIFICITY GASP-1 ELISA FOR DETECTING BREAST CANCER

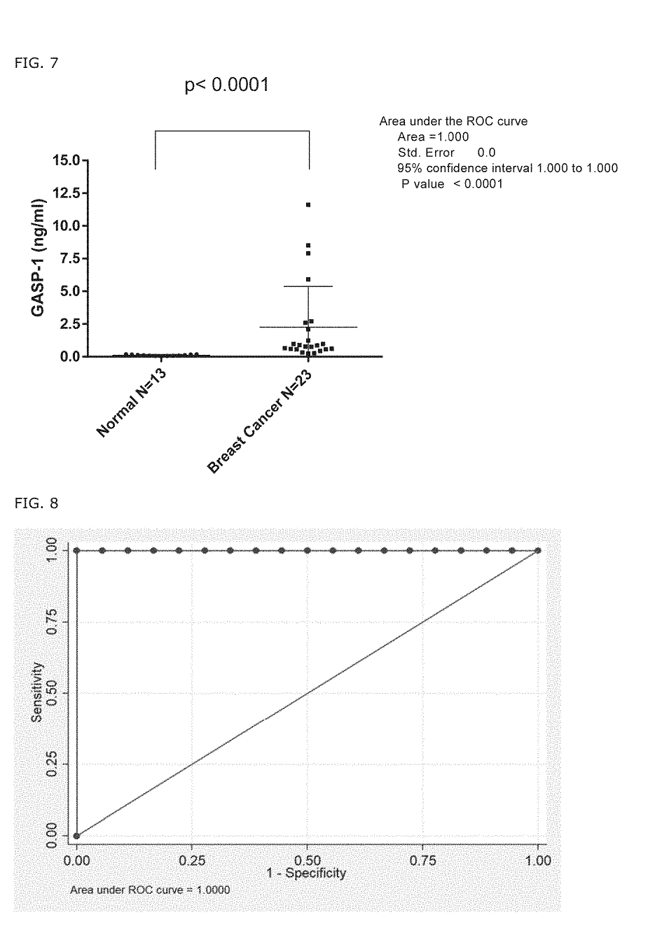

The competitive GASP-1 ELISA as described in Example 2 was used to quantity GASP-1 levels in serum samples from 18 symptom-free (normal) individuals and 37 breast cancer patients. As shown in FIG. 6, the average serum GASP-1 concentration for breast cancer patients was 3.5 ng/ml, which was about 13 times of the average serum GASP-1 concentration for normal individuals, which was only 0.27 ng/ml. Because there was a complete separation of GASP-1 levels between cancer patients and normal individuals, when a ROC (receiver operating characteristic) curve was constructed, the AUC (area under the curve) value was 1.0. An AUC value of 1.0 is a perfect number indicating 100% sensitivity and 100% specificity for the GASP-1 ELISA test. Some symptom-free (normal) individuals had very low serum GASP-1 levels at or above the detection limit of 0.01 ng/ml. In a separate 19 patient breast cancer patient analysis, we also found a clear separation between cancer patients and normal individuals (FIG. 7). Again, an AUC analysis shows 100% sensitivity and 100% specificity for this analysis (FIG. 8). No other breast cancer biomarker has been reported to have 100% sensitivity and 100% specificity. An AUC number of 1.0 suggests that GASP-1 is the best biomarker available for detecting breast cancer.

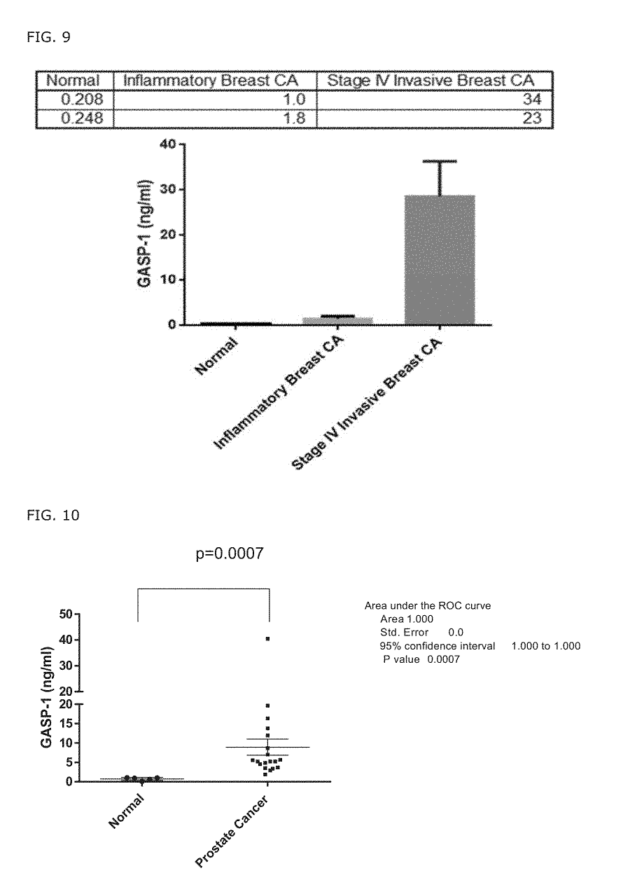

FIG. 9 shows that Stage 4 invasive breast cancer patients had extremely high GASP-1 levels, which are about 100 times or more when compared to those from normal individuals. Interestingly, another form of breast cancer, inflammatory breast cancer, which appears to be caused by viruses, does not over-produce GASP-1 (FIG. 9). This ELISA only detects cancers associated with overexpression of GASP-1.

### EXAMPLE 5. GASP-1 EXPRESSION IN PROSTATE CANCER AND BENIGN PROSTATIC HYPERPLASIA (BPH)

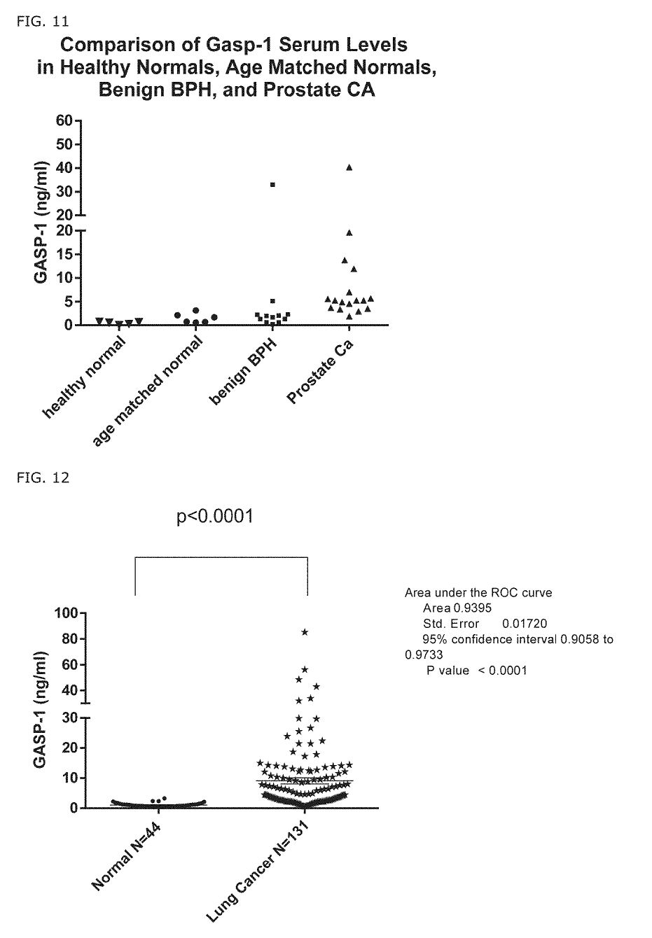

In a 19 patient prostate cancer study, a clear separation in GASP-1 levels between normal individuals and cancer patients was found (FIG. 10). The AUC number was 1.0, indicating 100% sensitivity and 100% specificity. As a comparison, a currently widely used PSA has a sensitivity of only 52% and a specificity of 79%. This means is that PSA has a false positive rate of 48% and false negative rate of 21%. Unlike the PSA test which cannot differentiate between prostate cancer and benign prostatic hyperplasia (BPH), our GASP-1 ELISA shows that BPH patients do not overexpress GASP-1 (FIG. 11). The high serum GASP-1 level in one BPH individual could be due to the presence of other cancer because GASP-1 is a pan-cancer biomarker. Therefore, o the present GASP-1 test in conjunction with the PSA test may be used to reduce the need for prostate biopsies due to the presence of high PSA value alone. By combining PSA test with GASP-1 test, only individuals with high levels of both PSA and GASP-1 will be required to have biopsy procedure. This would spare those individuals with high PSA but low GASP-1 level from undergoing biopsies.

### EXAMPLE 6. GASP-1 AS EXCELLENT LUNG CANCER BIOMARKER

Through collaboration with Lung Cancer Institute in Chengdu, China, a large population lung cancer clinical trial was conducted. Our results show that GASP-1 ELISA has an AUC value of 0.9395, which is better than all other reported lung cancer biomarkers (FIG. 12).

### EXAMPLE 7. GASP-1 AS AN EXCELLENT PANCREATIC CANCER BIOMARKER

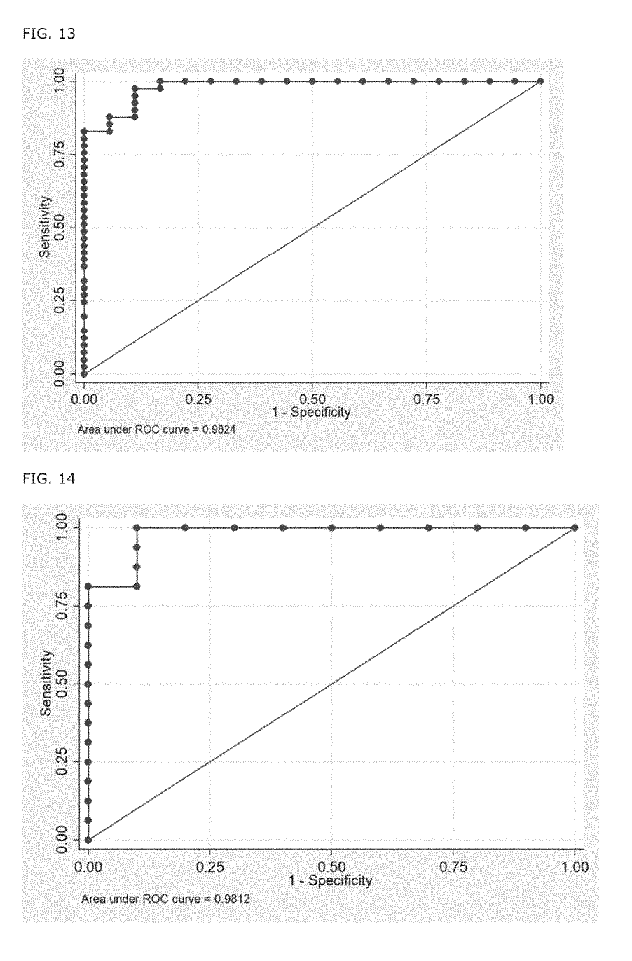

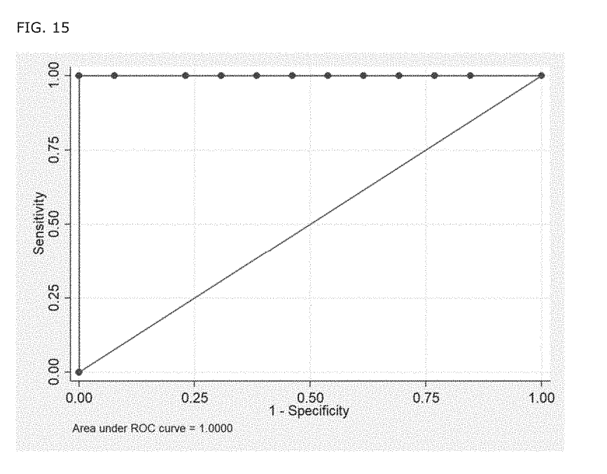

Similarly, when we conducted the ELISA analysis using 40 pancreatic cancer patients and 18 normal individuals, only very minor overlaps was observed between cancer patients and symptom-free (normal) individuals with an AUC number of 0.9824 (FIG. 13).

### EXAMPLE 8. GASP-1 AS AN EXCELLENT BIOMARKER FOR BOTH LIVER CANCER AND GLIOBLASTOMA

We also determined the GASP-1 levels between normal individuals and those with liver cancer and/or glioblastoma. FIG. 14 shows the analysis of 17 liver cancer patients and 10 symptom-free (normal) individuals using the competitive ELISA as described. An AUC value of 0.9812 indicates a sensitivity and specificity of over 95%. For glioblastoma, we obtained an AUC number of 1.0 indicating clear separation of GASP-1 levels between 10 symptom-free (normal) individuals and 12 glioblastoma patients (FIG. 1).

Because GASP-1 overexpression and their subsequently release as GASP-1 microvesicles or GASP-1 exosomes into circulation is cancer-specific, GASP-1 can be considered as a “universal” cancer biomarker. Such a universal cancer biomarker has been actively pursued by many but not discovered yet. As shown above, some of the cancer we tested such as breast, prostate and glioblastoma have an AUG number of 1.0 and one would also assume other cancers could also reach the perfect number. One reason for slightly tower AUC numbers in>some of the cancers we tested could be due to deficiencies in current assessment of normal individuals based solely on the appearance of symptoms. According to this criterion, a normal individual could mean that symptoms have not shown up in that individual. Because it normally takes many years for cancer symptoms to show up and during this latent period, many biochemical reactions leading to cancer may have already started. The finding that overexpression of GASP-1 is one of the required steps for cancer initiation and GASP-1 is only overexpressed in cancer cells means that we can detect very early stage cancer well before symptoms show up by looking at the appearance and increase in serum GASP-1 levels. We have also found that some symptom-free (normal) individuals are having serum GASP-1 levels that are at our limit of detection of 0.01 ng/ml indicating that most likely they are the healthiest individuals. We also found that there is only a very small range of serum GASP-1 levels in all the normal individuals we analyzed, beyond that would indicate the presence of early cancer. It is interesting to note that some older “normal” individuals (those at 70, or 80, or 85 years old) are having serum GASP-1 levels approaching the upper range of GASP-1 levels normally would be indicative of early stage cancer. These individuals may already have very early stage cancer but their symptoms have not been detected yet. Therefore, slightly higher serum GASP-1 levels in normal individuals could indicate very early cancer stage even though they are symptom-free. A serum GASP-1 scale can be proposed to indicate cancer severity similar to serum cholesterol test.

### EXAMPLE 9. USE OF SERUM GASP-1 LEVELS FOR ASSESSING THE EFFECTIVENESS OF CANCER TREATMENT

As cancer progresses, more GASP-1 is overexpressed an released. The competitive GASP-1, ELISA assay of Example 2 may be used to monitor the effectiveness of cancer therapy. Because the levels of serum GASP-1 or its peptide fragments in symptom-free (normal) individuals were very low whereas those in cancer patients were very highly elevated, it is likely that an effective cancer treatment would be reduction of serum GASP-1 or its fragments levels toward that of normal individuals. In a pilot study shown in Table 1, a pancreatic cancer patient with a serum GASP-1 level of 4.7 ng/ml was treated with 2 cycles of gemcitabine plus abraxane (gem/abraxane). The serum GASP-1 level dropped 65% from 4.7 to 1.6 ng/ml. After two additional cycles of treatment, the serum GASP-1 level dropped further to 0.4 ng/ml, which is approaching the level for a normal individual. Therefore, levels of serum GASP-1 or its fragments could be used to monitor the effectiveness of cancer treatment.

TABLE 1
GASP-1 levels can be used to monitor the effectiveness
of cancer treatment

| Sample | Serum GASP-1 levels (ng/ml) |
|---|---|
| Healthy Normal | 0.10 |
| Pancreatic CA patient | 4.7 |
| After 2 cycles of treatment | 1.6 |
| After 4 cycles of treatment | 0.4 |

Although the invention is illustrated and described herein with reference to specific embodiments, the invention is not intended to be limited to the details shown. Rather, various modifications may be made in the details within the scope and range of equivalents of the claims without departing from the invention.

What is claimed:

1. A method of detecting G protein coupled receptor-associated sorting 1 (GASP-1) or a fragment thereof in a sample, comprising:
(a) exposing a surface to a sample comprising GASP-1 or a fragment thereof, wherein a coating agent is immobilized to the surface and is selected from the group consisting of a conjugate of bovine serum albumin (BSA) and a GASP-1 peptide (BSA-GASP-1 conjugate), a capture antibody against a microvesicle or exosomal surface biomarker, and a combination thereof;
(b) immobilizing an anti-GASP-1 antibody to the surface via the coating agent in the presence of the GASP-1 or a fragment thereof;
(c) measuring the amount of the anti-GASP-1 antibody immobilized to the surface; and
(d) determining the presence of the GASP-1 or a fragment thereof in the sample based on the amount of the anti-GASP-1 antibody immobilized to the surface.

2. The method of claim 1, further comprising quantifying the concentration of the GASP-1 or a fragment thereof in the sample based on the amount of the anti-GASP-1 antibody immobilized to the surface and a standard curve of GASP-1 generated for the surface using the anti-GASP-1 antibody.

3. The method of claim 1, further comprising immobilizing the coating agent to a surface whereby the surface of step (a) is obtained.

4. The method of claim 1, wherein the coating agent is the BSA-GASP-1 conjugate, and wherein step (c) comprises binding the anti-GASP-1 antibody specifically to the BSA-GASP-1 conjugate.

5. The method of claim 1, wherein the GASP-1 peptide consists of the amino acid sequence of EEASPEAVAGVGFESK (SEQ ID NO: 2).

6. The method of claim 1, wherein the GASP-1 or a fragment thereof is on the surface of a microvesicle or an exosome in the sample and the coating agent is the capture antibody, wherein step (b) comprises binding the microvesicle or exosome specifically to the capture antibody, and wherein step (c) comprises binding the anti-GASP-1 antibody specifically to the GASP-1 or a fragment thereof.

7. The method of claim 1, wherein the microvesicle or exosomal surface biomarker is selected from the group consisting of CD9 CD63, CD81 and a combination thereof.

8. The method of claim 1, wherein the sample comprises 0.01-0.02 ng/ml of the GASP-1 or a fragment thereof.

9. The method of claim wherein the anti-GASP-1 antibody is selected from the group consisting of a recombinant monoclonal antibody, a recombinant polyclonal antibody, a humanized antibody and an antigen binding fragment thereof.

10. The method of claim 1, wherein the GASP-1 or a fragment thereof in the sample are from a biological fluid of a subject, further comprising determining the concentration of the GASP-1 or a fragment thereof in the biological fluid in the subject.

11. The method of claim 10, wherein the biological fluid comprises 0.01-0.02 ng/ml of GASP-1 or a fragment thereof.

12. The method of claim 10, wherein the biological fluid is selected from the group consisting of plasma, serum, saliva, urine, and cerebrospinal fluid.

13. The method of claim 10, wherein the biological fluid is serum.

14. The method of claim 10, wherein the subject is free of a cancer symptom.

15. The method of claim 10, wherein the subject has cancer.

16. The method of claim 10, wherein the subject has received a treatment of cancer, and wherein the biological fluid is in the subject after the treatment.

17. The method of claim 16, further comprising determining a concentration of the GASP-1 or a fragment thereof in a biological fluid in the subject before the treatment.

18. The method of claim 17, further comprising comparing the concentration of the GASP-1 or a fragment thereof in the biological fluid in the subject after the treatment with the concentration of the GASP-1 or a fragment thereof in the biological fluid in the subject before the treatment.

19. The method of any one of claims 14-16, wherein the cancer is selected from the group consisting of, breast cancer bladder cancer, ovarian cancer, colon cancer, endometrial carcinoma, esophagus squamous cell carcinoma, glioma, head cancer, neck cancer, hepatocellular carcinoma, infiltrating ductal breast carcinoma, larynx cancer, lung cancer, melanoma, mucinous cystadenocarcinoma of ovary, pancreatic cancer, prostate cancer, renal cell carcinoma, small bowel malignant stromal tumor, and stomach adenocarcinoma.

20. The method of any one of claims 14-16, wherein the cancer is selected from the group consisting of breast cancer, prostate cancer, lung cancer, pancreatic cancer, liver cancer and glioblastoma.

21. The method of claim 10, further comprising comparing the concentration of the GASP-1 or a fragment thereof in the biological fluid in the subject with a concentration of the GASP-1 or a fragment thereof in a control biological fluid in a symptom-free individual.

22. The method of claim 10, wherein the subject is suspected of having prostate cancer, further comprising comparing the concentration of the GASP-1 or a fragment thereof in the biological fluid in the subject with a concentration of the GASP-1 or a fragment thereof in a biological fluid in an individual having benign prostatic hyperplasia (BPH).

23. The method of claim 22, wherein the subject is tested positive in a prostate specific antigen (PSA) test.

24. The method of claim 10, wherein the subject is suspected of having stage IV invasive breast cancer, further comprising comparing the concentration of the to GASP-1 or a fragment thereof in the biological fluid in the subject with a concentration of the GASP-1 or a fragment thereof in a biological fluid in an individual having inflammatory breast cancer.

25. The method of any one of claims 1-24, further comprising detecting a cancer biomarker in the sample.

26. The method of claim 25, wherein the cancer biomarker is selected from the group consisting of CA125, CA19-9, CA15-3, and CEA.

27. A kit comprising:
(a) a surface, wherein a coating agent is immobilized to the surface and is selected from the group consisting of a conjugate of bovine serum albumin (BSA) and a GASP-1 peptide (BSA-GASP-1 conjugate), a capture antibody against a microvesicle or exosomal surface biomarker, and a combination thereof, and
(b) an anti-GASP-1 antibody.

28. The kit of claim 7, further comprising an instruction for carrying out the method of claim 1.

29. A kit comprising:
(a) a surface,
(b) a coating agent selected from the group consisting of a conjugate of bovine serum albumin (BSA) and a GASP-1 peptide (BSA-GASP-1 conjugate), a capture antibody against a microvesicle or exosomal surface biomarker, and a combination thereof, and
(c) an anti-GASP-1 antibody.

30. The kit of claim 29, further comprising a first instruction for immobilizing the coating agent to the surface and a second instruction for carrying out the method of claim 1.

31. The kit of claim 27 or 29, wherein the coating agent is the BSA-GASP-1 conjugate.

32. The kit of claim 27 or 29, wherein the GASP-1 peptide consists of the add sequence of EEASPEAVAGVGFESK (SEQ ID NO: 2).

33. The kit of claim 27 or 29, wherein the coating agent is the capture antibody.

34. The kit of claim 27 or 29, wherein the microvesicle or exosomal surface biomarker is selected from the group consisting of CD9, CD63, CD81 and a combination thereof.

35. The kit of claim 27 or 29, wherein the anti-GASP-1 antibody is biotinylated.


SEQUENCE LISTING (4 sequences)

SEQ ID NO: 1 - protein, 1395 aa, Homo sapiens
MTGAEIESGAQVKPEKKPGEEVVGGAEIENDVPLVVRPKVRTQAQIMPGARPKNKSKVMPGASTKVETSAVGGARPKSKAKAIPVSRFKEEAQMWAQPRFGAERLSKTERNSQTNIIASPLVSTDSVLVAKTKYLSEDRELVNTDTESFPRRKAHYQAGFQPSFRSKEETNMGSWCCPRPTSKQEASPNSDFKWVDKSVSSLFWSGDEVTAKFHPGNRVKDSNRSMHMANQEANTMSRSQTNQELYIASSSGSEDESVKTPWFWARDKTNTWSGPREDPNSRSRFRSKKEVYVESSSGSEHEDHLESWFGAGKEAKFRSKMRAGKEANNRARHRAKREACIDFMPGSIDVIKKESCFWPEENANTFSRPMIKKEARARAMTKEEAKTKARARAKQEARSEEEALIGTWFWATDESSMADEASIESSLQVEDESIIGSWFWTEEEASMGTGASSKSRPRTDGERIGDSLFGAREKTSMKTGAEATSESILAADDEQVIIGSWFWAGEEVNQEAEEETIFGSWFWVIDAASVESGVGVSCESRTRSEEEEVIGPWFWSGEQVDIEAGIGEEARPGAEEETIFGSWFWAENQTYMDCRAETSCDTMQGAEEEEPIIGSWFWTRVEACVEGDVNSKSSLEDKEEAMIPCFGAKEEVSMKHGTGVRCRFMAGAEETNNKSCFWAEKEPCMYPAGGGSWKSRPEEEEDIVNSWFWSRKYTKPEAIIGSWLWATEESNIDGTGEKAKLLTEEETIINSWFWKEDEAISEATDREESRPEAEEGDIIGSWFWAGEEDRLEPAAETREEDRLAAEKEGIVGSWFGAREETIRREAGSCSKSSPKAEEEEVIIGSWFWEEEASPEAVAGVGFESKPGTEEEEITVGSWFWPEEEASIQAGSQAVEEMESETEEETIFGSWFWDGKEVSEEAGPCCVSKPEDDEEMIVESWFWSRDKAIKETGTVATCESKPENEEGAIVGSWFEAEDEVDNRTDNGSNCGSRTLADEDEAIVGSWFWAGDEAHFESNPSPVFRAICRSTCSVEQEPDPSRRPQSWEEVTVQFKPGPWGRVGFPSISPFRFPKEAASLFCEMFGGKPRNMVLSPEGEDQESLLQPDQPSPEFPFQYDPSYRSVQEIREHLRAKESTEPESSSCNCIQCELKIGSEEFEELLLLMEKIRDPFIHEISKIAMGMRSASQFTRDFIRDSGVVSLIETLLNYPSSRVRTSFLENMIRMAPPYPNLNIIQTYICKVCEETLAYSVDSPEQLSGIRMIRHLTTTTDYHTLVANYMSGFLSLLATGNAKTRFHVLKMLLNLSENLFMTKELLSAEAVSEFIGLFNREETNDNIQIVLAIFENIGNNIKKETVFSDDDFNIEPLISAFHKVEKFAKELQGKTDNQNDPEGDQEN

SEQ ID NO: 2 - protein, 16 aa, Homo sapiens
EEASPEAVAGVGFESK

SEQ ID NO: 3 - protein, 13 aa, Homo sapiens
CSKSSPKAEEEEV

SEQ ID NO: 4 - protein, 14 aa, Homo sapiens
FWDGKEVSEEAGPC